(19) United States
(12) Patent Application Publication
(10) Pub. No.: US 2025/0288567 A1
(43) Pub. Date: Sep. 18, 2025

(54) PHARMACEUTICAL COMPOSITION FOR TREATING PRESBYOPIA

(71) Applicant: Sight Health Hong Kong Biotechnology Co., Limited, HONG KONG (HK)

(72) Inventor: Kang ZHANG, Del Mar, CA (US)

(21) Appl. No.: 19/028,919

(22) Filed: Jan. 17, 2025

Related U.S. Application Data
(60) Provisional application No. 63/566,502, filed on Mar. 18, 2024.

(51) Int. Cl. A61K 31/439; A61K 9/00; A61K 31/473; A61K 36/11; A61P 27/10

(52) U.S. Cl. CPC A61K 31/439; A61K 9/0048; A61K 31/473; A61K 36/11; A61P 27/10

(57) ABSTRACT

Disclosed is a pharmaceutical composition for treating presbyopia including a cholinesterase inhibitor in a therapeutically effective amount for treating presbyopia. Additionally, the pharmaceutical composition further includes an acetylcholinesterase inhibitor in a therapeutically effective amount for treating presbyopia. A method for treating presbyopia is also disclosed, including the following steps: administering a pharmaceutical composition including a cholinesterase inhibitor or a combination of a cholinesterase inhibitor and an acetylcholinesterase inhibitor to a patient in need thereof; and simultaneously detecting changes in reading distance (UNVA40) and computer screen usage distance (UNVA66) of the patient.

### CROSS-REFERENCE TO RELATED APPLICATIONS

The present application claims priority to U.S. Provisional Application No. 63/566,502, filed on Mar. 18, 2024.

### FIELD OF THE INVENTION

The present invention relates to the field of pharmacological treatment of presbyopia. More particularly, the present invention relates to pharmacological treatment of presbyopia by a cholinesterase inhibitor, an acetylcholinesterase inhibitor, and a combination thereof which shows a synergistic effect.

### BACKGROUND OF THE INVENTION

During youth years, the eye can easily adjust or increase the curvature of the lens by contracting the ciliary muscle. When concentrating on reading, the ciliary smooth muscle contracts, which relaxes the fibers of the zonular and makes the lens more rounded, thereby shortening the focal length of the eye to near distance.

Presbyopia is one of the manifestations of physical aging. With age, the lens gradually loses its normal progressive accommodation, resulting in an inability to focus sharply on objects at arm length or closer. Presbyopia is a physiological degeneration problem that almost everyone will eventually face, which significantly reduces quality of life and affect learning and work efficiency. Presbyopia reduces an individual's ability to perform visual tasks at close range, interfering with reading and using computer screens. This is a global problem that affects more than a billion people around the world. There are 390 million people over the age of 35 in China, of which: 146 million people suffer from moderate to severe presbyopia while eight million people suffer from severe presbyopia.

At present, the mainstream methods of correcting presbyopia are mainly wearing contact lenses or traditional glasses. Laser refractive surgery and intraocular lens implantation surgery can also improve presbyopia to a certain extent. However, these methods have their own shortcomings. The wearing of contact lenses is not easy and convenient, and may cause corneal epithelium damage, keratitis and other injuries. Long-term wearing of glasses increases visual fatigue, while laser refractive surgery and intraocular lens damage implantation surgery is invasive, with certain surgical risks such as intraocular infection, and even some surgical complications are irreversible.

Huperzine A eye drops are eye drops that are known to be safe, effective, and FDA-approved. Ophthalmic Huperzine A, used in the treatment of acute angle-closure glaucoma since the 1870s, reduces intraocular pressure and induces miosis by inducing miosis and accommodative spasm by stimulating muscarinic receptors in the iris and ciliary body. It is no longer the drug of choice for glaucoma due to the need for dosing every 6 hours and its potential for adverse effects at high doses. The most common adverse reactions are headache, brow pain, changes in adaptation, blurred vision, and eye irritation, but these discomforts are generally tolerated by the user with a low likelihood of permanent damage to the user's eye health.

There are a few existing compositions and methods in the market that assist in presbyopia treatment. Some of these examples are discussed in the following prior arts.

United States patent publication no. 20110152274A1 relates to the use of one or more parasympathomimetic drugs in combination with one or more alpha agonists to create optically beneficial miosis to, for example, temporarily treat presbyopia. The prior art provides a pharmaceutical preparation comprising a therapeutically effective amount of one or more parasympathomimetic drugs or cholinesterase inhibitors, or a pharmaceutically acceptable salt thereof, in combination with one or more alpha agonists or antagonists, or a pharmaceutically acceptable salt thereof. The prior art further provides for a method for treating, ameliorating or reducing presbyopia of a patient having an eye, comprising administering to said eye a pharmaceutically effective amount of the ophthalmic preparation. However, the use of parasympathomimetic drugs may lead to side effects such as blurred vision, headaches, or eye discomfort, particularly with prolonged use.

Canada patent publication no. 3140889A1 relates to the use of topical carbachol in combination with brimonidine to create optically beneficial miosis to temporarily treat presbyopia. A pharmaceutical formulation is used comprising a therapeutically effective amount of carbachol, or a pharmaceutically acceptable salt thereof, in combination with brimonidine, or a pharmaceutically acceptable salt thereof, specifically combined with permeation enhancers and excipients to increase efficacy and reduce ocular surface toxicity and improve tolerability. However, even with permeation enhancers, the combination of carbachol and brimonidine may still pose a risk of ocular surface toxicity, particularly with frequent use.

United States patent publication no. 20220175734A1 relates to using one or more parasympathomimetic drugs alone or together, or in combination with one or more alpha agonists to create optically beneficial miosis to temporarily create multifocality in a pseudophakic patient to treat presbyopia. A pharmaceutical preparation comprising a therapeutically effective amount of one or more parasympathomimetic drugs or cholinesterase inhibitors, alone or in combination with or a pharmaceutically acceptable salt thereof, in combination with one or more alpha agonists or antagonists, or a pharmaceutically acceptable salt thereof. A method for creating multifocality in a pseudophakic patient, reducing symptoms of presbyopia in a patient having an eye or both eyes through administering to an eye or eyes a pharmaceutically effective amount of the ophthalmic preparation is also disclosed. However, the risk of excessive miosis could lead to visual discomfort or complications, particularly in low-light conditions.

There is a significant need for new methods to alleviate presbyopia for patients who prefer not to undergo surgical interventions, such as intraocular lenses (IOLs) or laser ablation, or who wish to avoid corrective glasses. Additionally, it is essential to develop compositions that not only help improve near vision but also reduce any adverse effects associated with treatment, such as ocular surface toxicity or irritation. By focusing on these areas, the present invention can enhance patient comfort and satisfaction, ultimately addressing the unmet needs in the management of presbyopia.

### SUMMARY OF THE INVENTION

It is an objective of the present invention to provide a composition for treating presbyopia without invasive procedures.

It is also an objective of the present invention to provide a composition for improving reading distance (UNVA40) and computer screen usage distance (UNVA66) for patients with presbyopia.

It is further an objective of the present invention to provide to provide a composition that enhances tolerance to the adverse side effects of Huperzine A, such as headaches and eye pain, while also alleviating discomfort for some dry eye patients to a certain extent.

Accordingly, these objectives may be achieved by following the teachings of the present invention. The present invention relates to a pharmaceutical composition for treating presbyopia, comprising: a cholinesterase inhibitor in a therapeutically effective amount for treating presbyopia. Additionally, the pharmaceutical composition further comprising an acetylcholinesterase inhibitor in a therapeutically effective amount for treating presbyopia.

### BRIEF DESCRIPTION OF DRAWINGS

The features of the invention will be more readily understood and appreciated from the following detailed description when read in conjunction with the accompanying drawings of the preferred embodiment of the present invention.

FIG. 1A is a bar chart illustrating the improvement of UNVA40 and UNVA66 of all patients.

FIG. 1B is a scatter chart illustrating the improvement of UNVA40 and UNVA66 of all patients.

FIG. 2A is a bar chart illustrating the improvement of UNVA40 and UNVA66 of Group 1 (40 years old or below, 2 people with 4 eyes) patients.

FIG. 2B is a scatter chart illustrating the improvement of UNVA40 and UNVA66 of Group 1 (40 years old or below, 2 people with 4 eyes) patients.

FIG. 3A is a bar chart illustrating the improvement of UNVA40 and UNVA66 of Group 2 (41-50 years old, 9 people with 18 eyes) patients.

FIG. 3B is a scatter chart illustrating the improvement of UNVA40 and UNVA66 of Group 2 (41-50 years old, 9 people with 18 eyes) patients.

FIG. 4A is a bar chart illustrating the improvement of UNVA40 and UNVA66 of Group 3 (51-60 years old, 6 people with 12 eyes) patients.

FIG. 4B is a scatter chart illustrating the improvement of UNVA40 and UNVA66 of Group 3 (51-60 years old, 6 people with 12 eyes) patients.

FIG. 5A is a bar chart illustrating the improvement of UNVA40 and UNVA66 of Group 4 (61-70 years old, 2 people with 4 eyes) patients

FIG. 5B is a scatter chart illustrating the improvement of UNVA40 and UNVA66 of Group 4 (61-70 years old, 2 people with 4 eyes) patients.

FIG. 6A is a bar chart illustrating the improvement of UNVA40 and UNVA66 of Group 4 (over 70 years old, 1 person with 2 eyes) patients.

FIG. 6B is a scatter chart illustrating the improvement of UNVA40 and UNVA66 of Group 4 (over 70 years old, 1 person with 2 eyes) patients.

FIG. 7 is a bar chart illustrating the comparison of UNVA between the Group 2 (41-50 years old) and the Group 3 (51-60 years old).

FIG. 8 is a bar chart illustrating the UDVA improvement of 4 eyes with astigmatism.

FIG. 9 illustrates the molecular modelling of docking of cholinesterase (CE) inhibitors in the cholinesterase catalytic pocket.

### DETAILED DESCRIPTION OF THE INVENTION

For the purposes of promoting and understanding of the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

The present invention teaches a pharmaceutical composition for treating presbyopia, comprising: a cholinesterase inhibitor in a therapeutically effective amount for treating presbyopia.

In accordance with a preferred embodiment of the present invention, the cholinesterase inhibitor is Huperzine A (Huperzia serrata).

By constricting iris muscles with consequently constricting the pupil, cholinergic pathway agonists such as cholinesterase inhibitors can correct presbyopia. One of the agonists is Huperzine A. It improves uncorrected near vision by narrowing the pupil, creating a pinhole effect and increasing depth of focus.

In accordance with a preferred embodiment of the present invention, the Huperzine A (Huperzia serrata) is present at a concentration of about 0.1%-0.3%.

In accordance with a preferred embodiment of the present invention, the pharmaceutical composition further comprising an acetylcholinesterase inhibitor in a therapeutically effective amount for treating presbyopia.

In accordance with a preferred embodiment of the present invention, the cholinesterase inhibitor and acetylcholinesterase inhibitor are each present at a concentration of about 0.05%-0.3%.

In accordance with a preferred embodiment of the present invention, the acetylcholinesterase inhibitor is Tacrine (TRAC1).

In accordance with a preferred embodiment of the present invention, the pharmaceutical composition is formulated as an eye drop for topical administration.

The present invention also discloses a method for improving reading distance (UNVA40) and computer screen usage distance (UNVA66) for a patient, comprising administering to one eye of the patient a therapeutically effective amount of a cholinesterase inhibitor, or a combination of the cholinesterase inhibitor and an acetylcholinesterase inhibitor.

Considering the inconvenience caused by presbyopia to read and using computers in patients' life, the present invention also simultaneously detects the change data of reading distance (UNVA40) and computer screen usage distance (UNVA66).

The present invention further discloses a method for improving patient's distance vision with uncorrected distance visual acuity (UDVA) of less than 0.8 due to astigmatism during computer optometry, comprising administering to one eye of the patient a therapeutically effective amount of a cholinesterase inhibitor.

The present invention further details a method for treating presbyopia, comprising: administering a pharmaceutical composition comprising a cholinesterase inhibitor or a combination of a cholinesterase inhibitor and an acetylcholinesterase inhibitor to a patient in need thereof; and simultaneously detecting changes in reading distance (UNVA40) and computer screen usage distance (UNVA66) of the patient; discloses a synergistic effect of Huperzine A and TRAC1 in treatment of presbyopia.

In accordance with a preferred embodiment of the present invention, the administering of a pharmaceutical composition comprising a cholinesterase inhibitor to a patient in need thereof comprises the steps of: administering the pharmaceutical composition with the cholinesterase inhibitor at a concentration range of about 0.1%-0.3%; wherein the cholinesterase inhibitor comprises Huperzine A; and collecting and analyzing data on changes in visual acuity.

In accordance with a preferred embodiment of the present invention, the administering of a pharmaceutical composition comprising a combination of a cholinesterase inhibitor and an acetylcholinesterase inhibitor to a patient in need thereof comprises the steps of: administering the pharmaceutical composition with the cholinesterase inhibitor and acetylcholinesterase inhibitor each at a concentration range of about 0.05% to 0.3% to the patients respectively; wherein the cholinesterase inhibitor comprises Huperzine A and the acetylcholinesterase inhibitor comprises Tacrine; and collecting and analyzing data on changes in visual acuity.

### EXAMPLE

A clinical study shows the synergistic effects of Huperzine A and TRAC1 in treating presbyopia, with more details provided below. The present invention also discusses the efficacy and safety of different concentrations of Huperzine A eye drops for the topical treatment of presbyopia.

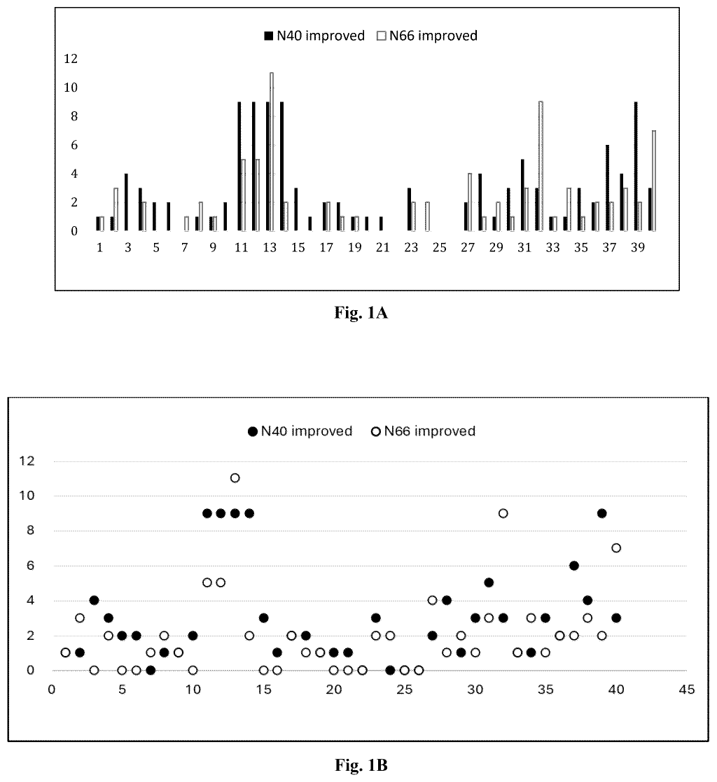

The following sections describe the present invention in more detail, referencing the illustrations in FIGS. 1A to 9.

A non-randomized clinical case study involving 40 eyes of 20 presbyopia patients with uncorrected distance visual acuity (UDVA) of 0.4-1.0 and uncorrected near visual acuity (UNVA) of less than Jaeger1, aged 33-70 years is conducted. The patients are randomly divided into 3 groups, using eye drops with concentration of Huperzine A ranging from 0.1%-0.3% for clinical trials. Data are collected on changes in visual acuity, including UDVA, UNVA40, UNVA66, pupil diameter changes, and non-contact intraocular pressure, before and 30 minutes after Huperzine A eye drops. The data are grouped according to drug concentration and age and are analyzed. The discomfort symptoms appeared half an hour after using the eye drops are recorded, and the time when the relevant symptoms disappeared is obtained during the telephone interview in the following 3 days. A detailed description of the case study is further illustrated below.

The clinical study of the present invention is conducted in March 2022 at a university hospital. Individuals over the age of 30 who feel their near vision has decreased are recruited and voluntarily participate in the test through a long-term open recruitment process. Inclusion criteria include best corrected distance visual acuity of 1.0, 40 cm reading visual acuity (UNVA40) or 66 cm computer visual acuity (UNVA66) lower than the Jaeger score J1, normal intraocular pressure, no history of retinal-related medical conditions, good general health, absence of systemic symptoms, and no rheumatic or systemic immune diseases.

Participants first complete a health declaration questionnaire with assistance from medical staff. They then measure their blood pressure and heart rate before undergoing computer optometry, as well as examinations for best corrected distance visual acuity and uncorrected distance visual acuity. They use the Jaeger score table to assess uncorrected reading visual acuity at 40 cm (UNVA40) and 66 cm (UNVA66) for computer visual acuity. Additionally, they have non-contact intraocular pressure measurements, pupil diameter assessments under room lighting, and a slit lamp examination by an experienced clinical ophthalmologist to rule out ocular diseases. Fundus photography is also performed.

Participants who meet the screening criteria are randomly divided into two groups. One drop of 0.1%-0.3% Huperzine A eye drops is administered in each eye. After 30 minutes, the best corrected distance visual acuity, uncorrected distance visual acuity at 40 cm (UNVA40), and 66 cm computer visual acuity (UNVA66) are measured again, along with non-contact intraocular pressure and pupil diameter under room lighting.

The drug used in this present test is Huperzine A at a range of 0.1%-0.3% concentration, using standard ophthalmic dropper bottles, each yielding 40 μL drops and each drop effectively dispenses 0.1%-0.3%.

The collected data are organized so that J1+ is recorded as 0 on the Jaeger scale, J1 as 1, J2 as 2, and so forth.

The patients are grouped by age: 40 years old or below (2 people, 4 eyes), 41-50 years old (9 people, 18 eyes), 51-60 years old (6 people, 12 eyes), 61-70 years old (2 people, 4 eyes), and over 70 years old (1 people, 2 eyes).

The comparison of the improvement in near vision before and after using Huperzine A eye drops is analyzed further below.

A total of 20 patients with 40 eyes are included, with an average age of 51.61 years. The average UNVA40 before treatment is J 5.50, decreasing to J 2.65 after treatment, resulting in an average improvement of J 2.85. The average UNVA66 before treatment is J 4.65, reducing to J 2.60 after treatment, with an average improvement of J 2.05, as shown in Table 1 below.

TABLE 1
Improvement of UNVA40 and UNVA66 of all patients

| Age | Near 40 | After | N40 improved | Near 66 | After | N66 improved |
|---|---|---|---|---|---|---|
| 51.61 | 5.50 | 2.65 | 2.85 | 4.65 | 2.60 | 2.05 |

FIG. 1A depicts a bar chart illustrating the improvement of UNVA40 and UNVA66 of the patients and FIG. 1B depicts a scatter chart illustrating the improvement of UNVA40 and UNVA66 of the patients.

Further, the performance of each age group is recorded and analyzed.

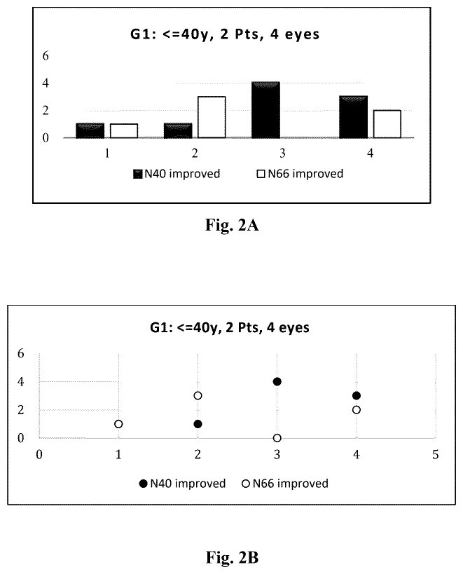

The results for Group 1, which includes patients of 40 years old or below (2 people with 4 eyes), are shown in Table 2 below, along with a bar chart (FIG. 2A) and a scatter chart (FIG. 2B) illustrating the improvement in UNVA40 and UNVA66.

Table 2 shows the results of 2 patients with an average age of 35.50 years. The average UNVA40 before treatment is J 3.25, decreasing to J 1.00 after treatment, resulting in an average improvement of J 2.85. Further, the average UNVA66 before treatment is J 4.25, reducing to J 2.75 after treatment, with an average improvement of J 2.05.

TABLE 2
Improvement of UNVA40 and UNVA66 of Group 1

| Age | Near 40 | After | N40 improved | Near 66 | After | N66 improved |
|---|---|---|---|---|---|---|
| 35.50 | 3.25 | 1.00 | 2.25 | 4.25 | 2.75 | 1.50 |

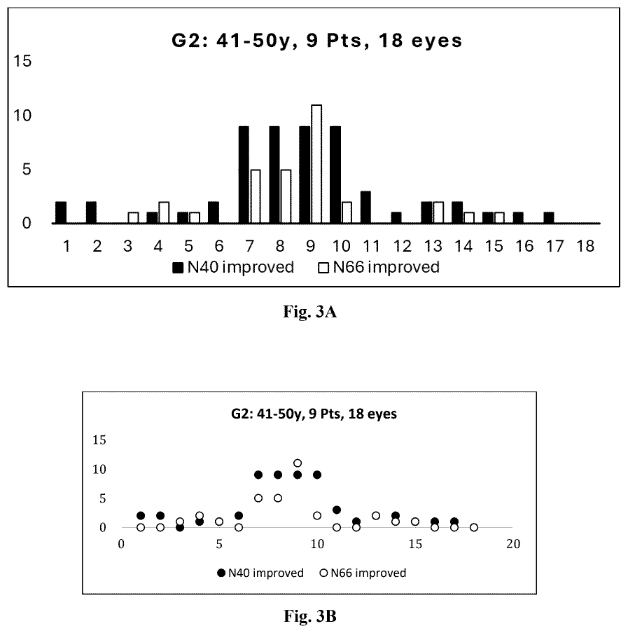

The results for Group 2, which includes patients aged 41-50 years old (9 people with 18 eyes), are shown in Table 3 below, along with a bar chart (FIG. 3A) and a scatter chart (FIG. 3B) illustrating the improvement in UNVA40 and UNVA66.

Table 3 shows the results of 9 patients with an average age of 47.67 years. The average UNVA40 before treatment is J 4.94, decreasing to J 1.89 after treatment, resulting in an average improvement of J 2.85. Further, the average UNVA66 before treatment is J 4.25, reducing to J 2.75 after treatment, with an average improvement of J 2.05.

TABLE 3
improvement of UNVA40 and UNVA66 of Group 2

| Age | Near 40 | After | N40 improved | Near 66 | After | N66 improved |
|---|---|---|---|---|---|---|
| 47.67 | 4.94 | 1.89 | 3.05 | 3.33 | 1.61 | 1.72 |

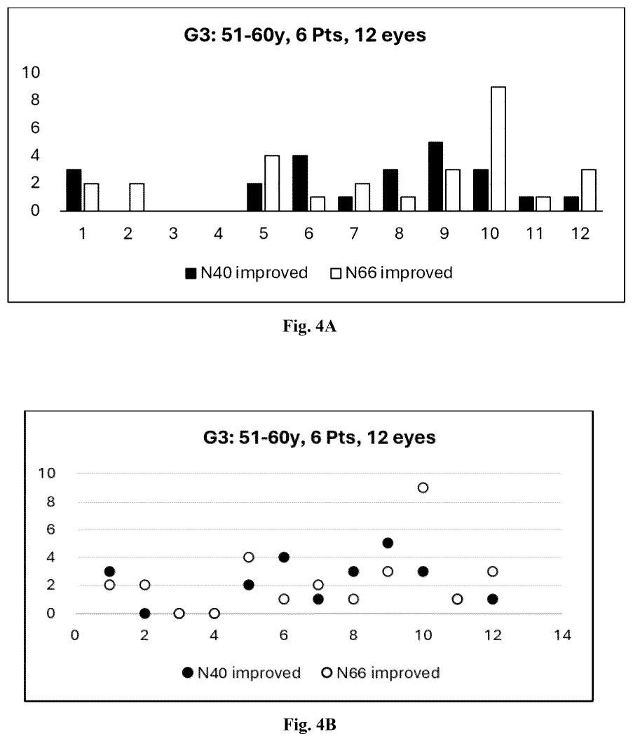

The results for Group 3, which includes patients aged 51-60 years old (6 people with 12 eyes), are shown in Table 4 below, along with a bar chart (FIG. 4A) and a scatter chart (FIG. 4B) illustrating the improvement in UNVA40 and UNVA66.

Table 4 shows the results of 6 patients with an average age of 57.00 years. The average UNVA40 before treatment is J 5.67, decreasing to J 3.75 after treatment, resulting in an average improvement of J 1.92. Further, the average UNVA66 before treatment is J 6.00, reducing to J 3.67 after treatment, with an average improvement of J 2.33.

TABLE 4
Improvement of UNVA40 and UNVA66 of Group 3

| Age | Near 40 | After | N40 improved | Near 66 | After | N66 improved |
|---|---|---|---|---|---|---|
| 57.00 | 5.67 | 3.75 | 1.92 | 6.00 | 3.67 | 2.33 |

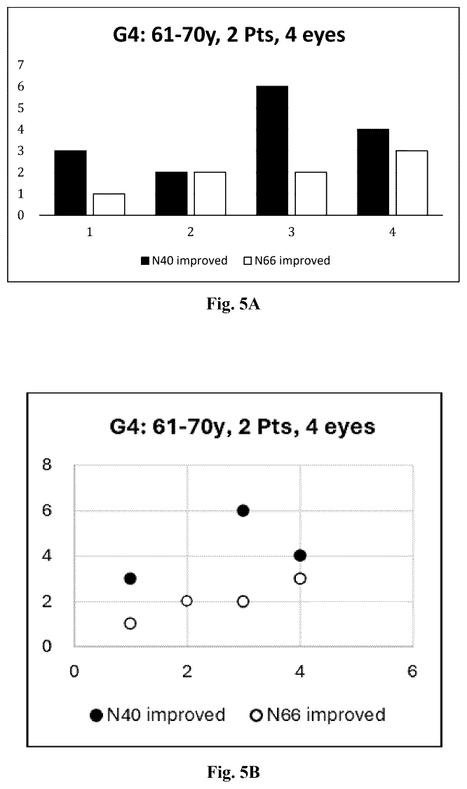

The results for Group 4, which includes patients aged 61-70 years old (2 people with 4 eyes), are shown in Table 5 below, along with a bar chart (FIG. 5A) and a scatter chart (FIG. 5B) illustrating the improvement in UNVA40 and UNVA66.

Table 5 shows the results of 2 patients with an average age of 62.00 years. The average UNVA40 before treatment is J 6.00, decreasing to J 2.25 after treatment, resulting in an average improvement of J 3.75. Further, the average UNVA66 before treatment is J 5.00, reducing to J 3.00 after treatment, with an average improvement of J 2.00.

TABLE 5
Improvement of UNVA40 and UNVA66 of Group 4

| Age | Near 40 | After | N40 improved | Near 66 | After | N66 improved |
|---|---|---|---|---|---|---|
| 62.00 | 6.00 | 2.25 | 3.75 | 5.00 | 3.00 | 2.00 |

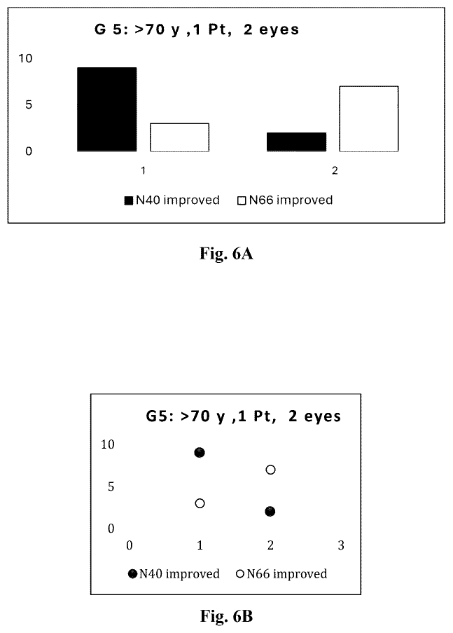

The results for Group 5, which includes a patient over 70 years old (1 person with 2 eyes), are shown in Table 6 below, along with a bar chart (FIG. 6A) and a scatter chart (FIG. 6B) illustrating the improvement in UNVA40 and UNVA66.

Table 6 shows the results of 2 patients with an average age of 72.00 years. The average UNVA40 before treatment is J 13.00, decreasing to J 7.00 after treatment, resulting in an average improvement of J 6.00. Further, the average UNVA66 before treatment is J 8.50, reducing to J 4.00 after treatment, with an average improvement of J 4.50.

TABLE 6
Improvement of UNVA40 and UNVA66 of Group 5

| Age | Near 40 | After | N40 improved | Near 66 | After | N66 improved |
|---|---|---|---|---|---|---|
| 72.00 | 13.00 | 7.00 | 6.00 | 8.50 | 4.00 | 4.50 |

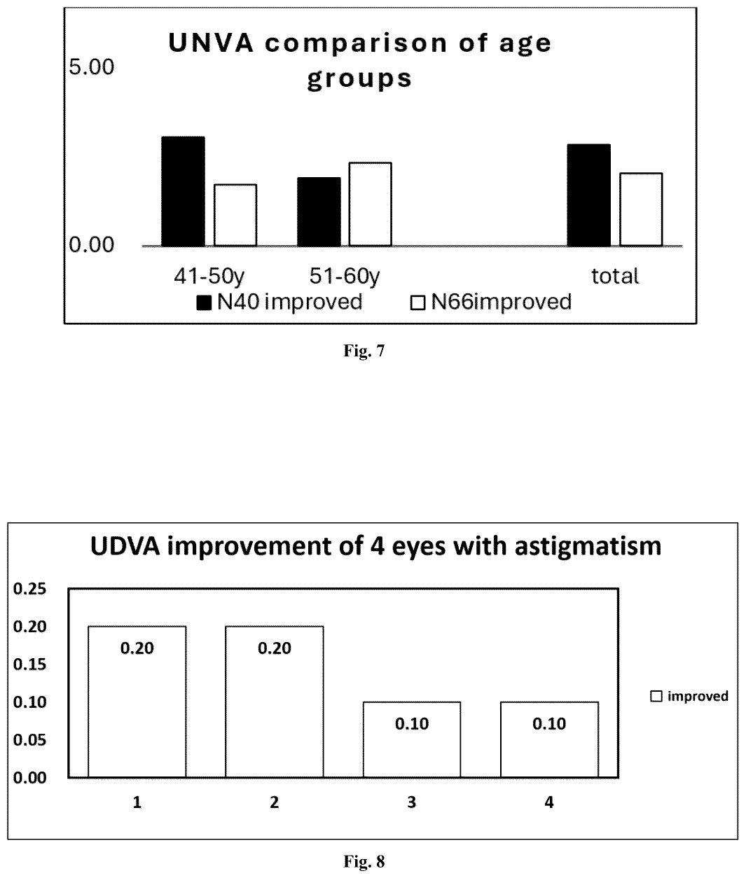

Additionally, the comparison among different age groups is made. Group 2 (41-50 years old) is compared with Group 3 (51-60 years old), showing that A has a more significant effect on the former group. The comparison of UNVA between the 41-50-year-old group and the 51-60-year-old group is shown in Table 7, along with a bar chart (FIG. 7).

TABLE 7
Comparison of UNVA improvement between Group 2 and Group 3

| Age Group | Eyes | N40 improved | N66 improved |
|---|---|---|---|
| 41-50 years old | 18 | 3.06 | 1.72 |
| 51-60 years old | 12 | 1.92 | 2.33 |
| Total | | 2.85 | 2.05 |

Furthermore, it is found in the present invention that when the patient's UDVA is less than 0.8 due to astigmatism during computer optometry, Huperzine A also has a certain effect on improving the distance vision. The present invention selects 2 patients with a total of 4 eyes with astigmatism. After using Huperzine A eye drops, the UDVA increases by 0.2 and 0.1 respectively. This is shown in FIG. 8.

Based on a clinical study above, which involves a total of 20 patients with a mean age at baseline of 51.90 years (range, 33-72 years), where the baseline UNVA (40) is 5.50 Jaeger, and UNVA (66) is 4.65 Jaeger, it is shown that the present Huperzine A has a significant effect on near vision, with an average improvement of 2.85 Jaeger in UNVA40 in all groups, and an average improvement of 2.05 Jaeger in UNVA66.

The effects of different concentrations of Huperzine A eye drops on near vision are all obvious across different age groups. The improvement of vision is more significant in the reading distance UNVA40 or the computer screen using distance UNVA66. In different age groups, the 41-50-year-old group is compared with the 51-60-year-old group, of which Huperzine A has a more significant effect on the former group.

In addition, it is found in the present invention that when the patient's UDVA is less than 0.8 due to astigmatism during computer optometry, Huperzine A also has a certain effect on improving the distance vision. The present invention selects 2 patients with a total of 4 eyes with astigmatism. After using Huperzine A eye drops, the UDVA increases by 0.2 and 0.1 respectively. This shows that Huperzine A can also improve the UDVA of patients with astigmatism to a certain extent.

In this present invention, the main side effects reported by the patients include headache, dizziness, eye pain, and decreased light perception. The duration of these side effects varies from 2 to 5 hours and gradually relieves over time. Additionally, some patients report an increase in tear secretion after using the eye drops.

In addition, due to the pinhole effect, the adverse side effects of Huperzine A, such as headache and eye pain, can be tolerated and gradually relieve over time. Furthermore, another side effect of Huperzine A—lacrimation (6%)—can help alleviate discomfort for some patients with dry eyes to a certain extent.

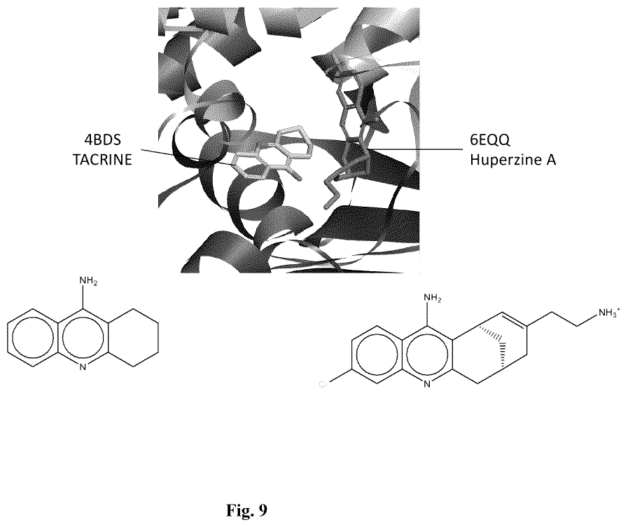

Furthermore, the synergistic effect of Huperzine A and Tacrine (TRAC1) is demonstrated below. Molecular modeling of docking of CE inhibitors in the cholinesterase catalytic pocket as shown in FIG. 9 is performed, which shows that both Huperzine A and TRAC1 binds to close yet distinct sites within the cholinesterase catalytic pocket. This suggests a potential synergistic effect of the two drugs. This hypothesis is tested by using 0.05%-0.3% Huperzine A and 0.05%-0.3% TRAC1. Three patients are treated with each drug, as well as the combination of both drugs. Table 8 presents the visual acuity readings before and after treatment with Huperzine A, TRAC1, and their combination. Based on the results in Table 8, it is concluded that there is a synergistic effect when using Huperzine A in conjunction with TRAC1, leading to improved UDVA.

TABLE 8
Visual acuity reading for before and after treatment

| | | Near 40 cm | | | Near 66 cm | | |
|---|---|---|---|---|---|---|---|
| | Drug(s) | Before | After | | Before | After | |
| | used | treatment | treatment | Improvement | treatment | treatment | Improvement |
| Patient 1 | | | | | | | |
| Right eye | Huperzine A | 6 | 3 | 3 | 4 | 2 | 2 |
| (OD) | Tacrine | 6 | 3 | 3 | 4 | 2 | 2 |
| | Both | 6 | 1 | 5 | 4 | 1 | 3 |
| Left eye | Huperzine A | 5 | 3 | 2 | 4 | 2 | 2 |
| (OS) | Tacrine | 5 | 3 | 2 | 4 | 2 | 2 |
| | Both | 5 | 1 | 4 | 4 | 1 | 3 |
| Patient 2 | | | | | | | |
| Right eye | Huperzine A | 10 | 4 | 6 | 6 | 3 | 3 |
| (OD) | Tacrine | 10 | 4 | 6 | 6 | 3 | 3 |
| | Both | 10 | 2 | 8 | 6 | 1 | 5 |
| Left eye | Huperzine A | 9 | 4 | 5 | 6 | 2 | 4 |
| (OS) | Tacrine | 9 | 4 | 5 | 6 | 2 | 4 |
| | Both | 9 | 1 | 8 | 6 | 1 | 5 |
| Patient 3 | | | | | | | |
| Right eye | Huperzine A | 8 | 2 | 6 | 7 | 2 | 5 |
| (OD) | Tacrine | 8 | 2 | 6 | 7 | 2 | 5 |
| | Both | 8 | 1 | 7 | 7 | 1 | 6 |
| Left eye | Huperzine A | 11 | 3 | 8 | 9 | 3 | 6 |
| (OS) | Tacrine | 11 | 3 | 8 | 9 | 3 | 6 |
| | Both | 11 | 1 | 10 | 9 | 1 | 8 |

The miotic effect of Huperzine A provides a new safe and non-invasive possibility for the medical treatment of presbyopia, which produces a pinhole effect and increases the depth of focus, thereby improving uncorrected near vision.

The present invention shows that Huperzine A eye drops with a concentration ranging from 0.1%-0.3% have significant effects on improving the near vision of different age groups. Comparing the data of different age groups, Huperzine A's effectiveness on relatively young presbyopia or early presbyopia is more pronounced, suggesting that Huperzine A can benefit more in the early stages of presbyopia.

The present invention also shows that Huperzine A and Tacrine combination at a range of 0.05%-0.3% each has a better result than either alone, indicating a synergistic effect.

In addition, due to pinhole effect, Huperzine A also improves the UDVA of patients with astigmatism to a certain extent, the principle of which may be attributed to the reduction of incidence of light entering through the cornea with astigmatism.

The patients who participate in this present invention do not report suffering from obvious adverse effect with the use of Huperzine A, such as headaches or eye discomfort, and the use of Huperzine A does not significantly impact their daily lives. Additionally, some patients report that after using the eye drops, they experience increased tear secretion, which helps alleviate some discomfort caused by dry eye syndrome to a certain extent.

The present invention explained above is not limited to the aforementioned embodiment and drawings, and it will be obvious to those having an ordinary skill in the art of the prevent invention that various replacements, deformations, and changes may be made without departing from the scope of the invention.

1. A pharmaceutical composition for treating presbyopia, comprising:
a cholinesterase inhibitor in a therapeutically effective amount for treating presbyopia.

2. The pharmaceutical composition according to claim 1, wherein the cholinesterase inhibitor is Huperzine A (Huperzia serrata).

3. The pharmaceutical composition according to claim 2, wherein the Huperzine A (Huperzia serrata) is present at a concentration of about 0.1%-0.3%.

4. The pharmaceutical composition according to claim 1, wherein the pharmaceutical composition further comprising an acetylcholinesterase inhibitor in a therapeutically effective amount for treating presbyopia.

5. The pharmaceutical composition according to claim 4, wherein the cholinesterase inhibitor and acetylcholinesterase inhibitor are each present at a concentration of about 0.05%-0.3%.

6. The pharmaceutical composition according to claim 5, wherein the acetylcholinesterase inhibitor is Tacrine (TRAC1).

7. The pharmaceutical composition according to claim 1, wherein the pharmaceutical composition is formulated as an eye drop for topical administration.

8. A method for improving reading distance (UNVA40) and computer screen usage distance (UNVA66) for a patient, comprising administering to one eye of the patient a therapeutically effective amount of a cholinesterase inhibitor, or a combination of the cholinesterase inhibitor and an acetylcholinesterase inhibitor.

9. A method for improving patient's distance vision with uncorrected distance visual acuity (UDVA) of less than 0.8 due to astigmatism during computer optometry, comprising administering to one eye of the patient a therapeutically effective amount of a cholinesterase inhibitor.

10. A method for treating presbyopia, comprising:
administering a pharmaceutical composition comprising a cholinesterase inhibitor or a combination of a cholinesterase inhibitor and an acetylcholinesterase inhibitor to a patient in need thereof; and
simultaneously detecting changes in reading distance (UNVA40) and computer screen usage distance (UNVA66) of the patient.

11. The method according to claim 10, wherein the administering of a pharmaceutical composition comprising a cholinesterase inhibitor to a patient in need thereof comprises the steps of:
administering the pharmaceutical composition with the cholinesterase inhibitor at a concentration range of about 0.1%-0.3%;
wherein the cholinesterase inhibitor comprises Huperzine A; and
collecting and analyzing data on changes in visual acuity.

12. The method according to claim 10, wherein the administering of a pharmaceutical composition comprising a combination of a cholinesterase inhibitor and an acetylcholinesterase inhibitor to a patient in need thereof comprises the steps of:
administering the pharmaceutical composition with the cholinesterase inhibitor and acetylcholinesterase inhibitor each at a concentration range of about 0.05% to 0.3% to the patients respectively;
wherein the cholinesterase inhibitor comprises Huperzine A and the acetylcholinesterase inhibitor comprises Tacrine; and
collecting and analyzing data on changes in visual acuity.
